(19) United States
(12) Patent Application Publication
(10) Pub. No.: US 2024/0263253 A1
(43) Pub. Date: Aug. 8, 2024

(54) DISCOVERY METHOD OF COTTON SALT-TOLERANT GENES BASED ON COMBINED TRANSCRIPTOME AND PROTEOME ANALYSIS AND USE THEREOF

(71) Applicant: Nantong University, Nantong (CN)

(72) Inventors: Baohua Wang, Nantong (CN); Kangtai Sun, Nantong (CN); Zhimin Zhuang, Nantong (CN); Hui Fang, Nantong (CN); Kai Wang, Nantong (CN); Jinlei Han, Nantong (CN); Meijun Ji, Nantong (CN); Wenxiang Feng, Nantong (CN)

(21) Appl. No.: 18/256,472

(22) PCT Filed: Apr. 20, 2023
(86) PCT No.: PCT/CN2023/089399

§ 371 Date: Jun. 8, 2023

(30) Foreign Application Priority Data
Apr. 27, 2022 (CN) 202210456826.3

(51) Int. Cl. C12Q 1/6895; C12N 15/10; C12N 15/82; C12Q 1/6851

(52) U.S. Cl. CPC C12Q 1/6895; C12N 15/1096; C12N 15/8218; C12N 15/8273; C12Q 1/6851; C12Q 2600/13

(57) ABSTRACT

The present disclosure relates to the technical field of plant genetics, particularly to a discovery method of cotton salt-tolerant genes based on combined transcriptome and proteome analysis and use thereof. In the present disclosure, upland cotton salt-tolerant variety Tongyan No. 1 serving as a material is subjected to transcriptome and proteome sequencing under salt stress and control; salt-tolerant candidate genes are preliminarily determined via the combined analysis and salt-tolerant differential genes are subjected to reverse transcription-quantitative polymerase chain reaction (RT-qPCR) expression analysis, and finally function validation is conducted in cotton. The two cotton salt-tolerate genes Gh_D10G0907 and Gh_D11G0978 obtained in the present disclosure are respectively located on cotton chromosomes D10 and D11; the molecular marker-assisted breeding of salt-tolerate cotton varieties can be achieved by utilizing the two genes.

### TECHNICAL FIELD

The present disclosure relates to the technical field of plant genetics, particularly to a discovery method of cotton salt-tolerant genes based on combined transcriptome and proteome analysis and use thereof.

### BACKGROUND

Approximately 20% of world's soil is affected by salinization, and the trend is worsening. More half of the arable land is expected to be salinized by 2050, seriously wasting large areas of land and severely affecting crop production. Due to being confused easily, the saline land and alkaline land are collectively referred to as saline land. In fact, the main characteristics of soil salinization are increases in both salinity and pH value, rather than single non-biotic stress alone. Over-high salinity leads to severe damage or even death to plants. Cultivating salt-tolerant plant varieties and improving the salt tolerance of plants are effective biological methods to utilize the saline land; and meanwhile, good ecological benefits and economic benefits are generated, thereby promoting the sustained development of agriculture.

Transcriptome sequencing is a method for researching differential gene expression, functional annotation, metabolic regulation prediction and SNP site detection; it has been widely applied to cotton research at present. The great progress of cotton genome sequencing has laid the foundation for gene mining and functional annotation. The comprehensive use of transcriptome sequencing based on a high-throughput second-generation sequencing technology can more quickly determine candidate genes and explore the molecular mechanism of cotton salt tolerance.

Proteomics is a study on all proteins contained in tissues and cells. A proteomic technology system provides a possibility to comprehensively study related metabolic processes and signal pathways of plants under salt stress. With the increasing maturation of genome research development, plant proteomics has also gained great progress. Differential proteomics mainly functions as studying specific protein expression from functional modes, and becomes an important research method and tool for plant physiology, development and genetics. Identifying the differences and changes of proteomes between different varieties or different states of plant samples and studying the performance of plants under stress using differential proteomics and their adaptation methods provide the possibility for analyzing the response of plants to stress.

With the increasing wide use of post genomics, high-throughput omics research methods and analysis techniques such as transcriptome and proteomics become increasing mature, and the combined analysis method has been widely applied to the field of plant research. Due to the interconnected nature of organisms and their environments, ordinary research cannot fully explain the response of plants to the environment in a short period of time; the combined transcriptome and proteome analysis can more completely understand the mutual regulation mechanism of proteins and transcript levels and know the complex physiological and biochemical reactions inside the organisms so as to link the internal laws of life activities with the external environment.

The tetraploid upland cotton “Tongyan No. 1” selected in the early stage of the present application has shown strong salt tolerance and was suitable for planting in low-salinity saline land. In the present application, combined transcriptome and proteomics analysis is performed on “Tongyan No. 1” under salt stress, and key genes and important pathways corresponding to salt stress in cotton are identified, which is conducive to the exploration and molecular marker development of cotton salt-tolerant candidate genes, and is of great significance for breeding salt-tolerant cotton varieties and developing and utilizing saline land.

### SUMMARY

The objective of the present disclosure is to provide a discovery method of cotton salt-tolerant genes based on combined transcriptome and proteome analysis and use thereof in order to solve the defects existing in the prior art.

To achieve the above objective, the present disclosure adopts the following technical solution:

Provided is a discovery method of cotton salt-tolerant genes based on combined transcriptome and proteome analysis, where the cotton salt-tolerant genes include cotton salt-tolerant gene Gh_D10G0907 and cotton salt-tolerant gene Gh_D11G0978 which are respectively located on cotton chromosomes D10 and D11.

Preferably, the cDNA sequence information of the cotton salt-tolerant gene Gh_D10G0907 and the cotton salt-tolerant gene Gh_D11G0978 is as follows:

>Gh_D10G0907.1
ATGGCTTCAGTGAATCTGGGCTCTTTGGTTCAATCATACTCCATTTTCA
ACCAAGCTTCC
AGAAACAATTCCAAGTCTTTTCCTTTACCCAGATCCTTTCCCTTCAATC
CCCTCAAGAAT
ATCTCATCTTCAACTCCCAAAAGACGTAGCTTTACATCTCTTCCTTCGA
TATCCTCAGTC
CTTACCAAAGAAGACGCGGTCATGGAAGAAGACCAAGACCCTCAATTAC
CAAGTTTTGAT
TTCAAATCATTCATGATACACAAGGGTAATGCAGTTAACCAAGCCCTGG
ACTCGGCTGTT
CCACTCCGTGACCCTGTTAAAATCCATGAAGCAATGCGTTACTCCCTTT
TAGCCGGTGGC
AAAAGGGTCCGCCCAGTTCTTTGTTTGGCTGCTTGTGACCTTGTTGGTG
GCAAAGAATCC
ATGGTTATGCCAGCAGCTTGTGCTCTTGAAATGATCCACACCATGTCTT
TAGTCCACGAT
GATCTTCCTTGCATGGACAACGATGATCTTCGTAGAGGGAAACCAACTA
ACCACAAAGTT
TATGGTGAAGATATAGCTGTGTTAGCAGGGGATGCTCTTTTAGCCTTTT
CGTTTGAACAT
ATAGCTGTATCCACAGTTGGTGTCACACCTGATAGGATTGTAAGAGCAA
TTGGGGAATTA
GCTAAATCTATTGGGGCTGAAGGGTTGGCGGCTGGTCAAGTTGTGGATA
TAACCAGTGAG
GGTCTAACCAATGTGGGGTTGGATCATTTAGAATTCATTCATGTTCATA
AAACTGCTCCA
TTGCTTGAAGCAGCTGCGGTTTTAGGGGCTATTCTTGGAGGTGGACATG
ATGAAGATGTG
GAAAAGTTGAGGAAATTTGCAAGGAATATTGGGCTTTTATTTCAAGTTG
TGGATGATATT
CTTGATGTAACAAAGTCATCTAAAGAATTAGGGAAGACTGCAGGGAAAG
ATTTGGTGGCT
GATAAAGTGACTTATCCTAAATCGATGGGGATAAACAAATCAAAGGAGT
TTGCAGAGAAG
TTGAAGAGTGATGCATTAGAGTTGCTTCAAGGGTTTGATCCTGAGAAAT
CTACCCCCTTA
ATTGCTTTAGCTAATTATATAGCTTACAGGCAAAATTAG
>Gh_D11G0978.1
ATGTCGCAGTTGTTGGAGAAGGCGAAGGACTTCGTGGTGGATAAGGTGG
CCAACATAAAG
AAGCCGGAGGCTAGTGTCTCGGACGTTGATCTGAAACATGTGAGCCGTG
AGTGCGTCGAG
TATGGCGCTAAGGTCTCTGTCTCCAACCCCTACAGCCATTCCATCCCCA
TTTGTGAGATC
TCTTACAATTTCAAAAGTGCTGGAAGAGGGATAGCATCAGGGACAATAC
CAGACCCGGGG
TCATTGAAAGCCGGCGACACAACGATGCTGGACGTGCCAGTGAAGGTGC
CGTATAACATC
TTAGTAAGCTTGGCAAAGGATATTGGTGCAGATTGGGACATTGACTATG
AATTGGAATTG
GGTCTCACCATTGATCTTCCTATCATGGGGAACTTCACTATCCCTCTCT
CTCAGAAAGGA
GAGATCAAGCTTCCTACTCTCAGTGACATCTTTTAG

Preferably, the discovery method of cotton salt-tolerant genes based on combined transcriptome and proteome analysis includes the following steps:

Step 1, transcriptome and proteome sequencing and combined analysis

Salt-tolerant cotton varieties are planted, and 6 seedlings with good growth status and similar morphology are selected and divided into two groups when the cotton seedlings grow to the four-leaf stage under the same environment, where one group is a control group, and the other group is an experimental group. The experimental group is irrigated with 200 ml of 250 mmol/l NaCl solution at 5 pm every day; the control group is irrigated with an equal amount of distilled water. After two days, main leaves of three seedlings in the control group are respectively marked as CK1, CK2 and CK3, and main leaves of three seedlings in the experimental group are also respectively marked as Salt1, Salt2 and Slat3. Finally, 6 samples are frozen in dry ice, and then RNA is respectively extracted for transcriptome sequencing, and proteins are respectively extracted for proteome sequencing.

A total of 24 commonly differentially expressed genes are obtained by combined transcriptome and proteome analysis, and all the 24 genes exhibit significant differential expression in transcriptome and proteome results. Two significantly differentially-expressed genes Gh_D10G0907 and Gh_D11G0978 are obtained through annotation and alignment of target genes, comparison of expression difference before and after salt stress and summarization of gene functions, and their homologous genes in other species are found with significant salt tolerance. Glycerol-3-phosphate and ADP-glucose are synthesized to obtain glycerol glucose phosphate by using Gh_D10G0907 as glycerol glucoside phosphate synthase (GGPS) which is a known salt-tolerant gene in algae and microorganisms. The Gh_D10G0908 gene is a class of late embryogenesis abundant (LEA) protein related genes, also participates in regulating permeation and a class of known stress genes, which is found to have slat-tolerant, drought-tolerant, and cold-tolerant functions in multiple crops such as corns and wheat. Hence, these two genes are determined as target genes for salt-tolerant function validation.

Step 2, expression mode and function validation of salt-tolerant genes

Step 2.1, primer design and synthesis are performed on the above salt-tolerant candidate genes by using Primer5, reverse transcription of RNA into cDNA is performed, and expression mode validation is performed by utilizing real-time quantitative PCR;

Step 2.2, gene cloning of candidate genes and functional validation based on virus-induced gene silencing (VIGS) are performed in cotton. Gh_D10G0907 has 372 full-length 1119 bp coding amino acids, and belongs to GGPS family. Gh_D10G0908 has 151 full-length 456 bp coding amino acids, and belongs to LEA family. Both of the two families have been validated for salt tolerance in different species. The silenced plants of Gh_D10G0907 and Gh_D10G0908 are obtained by using a VIGS technology, and the silenced plants have early wilting and significant salt damage phenotype after salt treatment; moreover, the silenced plants have higher levels of reactive oxygen species (ROS) than the control group, and therefore the two genes are believed to have pivotal role in salt stress in upland cotton.

The present disclosure also provides use of the cotton salt-tolerant gene Gh_D10G0907 and the cotton salt-tolerant gene Gh_D11G0978 obtained by using the above discovery method in molecular marker-assisted breeding of cotton varieties.

Compared with the prior art, the present disclosure has the beneficial effects:

In the present disclosure, the salt-tolerant cotton variety Tongyan No. 1 is used as a material, transcriptome sequencing and proteome sequencing are carried out under salt stress and control, salt-tolerant candidate genes are preliminarily determined through combined analysis, and RT-qPCR expression analysis is carried out on salt-tolerant differential genes, finally VIGS functional validation is carried out in cotton, and the finally obtained cotton salt-tolerant genes Gh_D10G0907 and Gh_D11G0978 are respectively located on chromosomes D10 and D11; the genes can be transferred to cotton materials with excellent agronomic traits through molecular marker-assisted breeding, thereby developing cotton materials or varieties with salt tolerance and excellent agronomic traits.

### BRIEF DESCRIPTION OF THE DRAWINGS

FIG. 1 is a RT-qPCR expression validation result graph of differential expression salt-tolerant gene under salt stress according to the present disclosure.

FIG. 2 is a differential expression comparison graph of four groups of cottons under salt stress in a VIGS validation experiment according to the present disclosure.

Note: pTRV: 00 is the empty vector representative of negative control; pTRV: GhCLA is the vector transferred with GhLCA1, i.e., cotton Cloroplastos alterados 1, GhCLA1, 420 bp; after GhCLA silencing, cotton leaves exhibit an albino phenotype; therefore, plants with silencing GhCLA genes are used as positive control. pTRV: Gh_D10G0907 and pTRV: Gh_D11G0978 representative of experiment groups (Gh_D10G0907 and Gh_D11G0978 genes are transferred).

### DETAILED DESCRIPTION OF THE EMBODIMENTS

Next, the technical solution in embodiments of the present disclosure will be clearly and completely described in combination with drawings to make those skilled in the art better understand the advantages and features of the present disclosure, thereby clearly defining the scope of protection of the present disclosure. The embodiments described herein are only some embodiments of the present disclosure but not all the embodiments. Based on the embodiments of the present disclosure, other embodiments obtained by persons of ordinary skill in the art without contributing creative efforts are all included within the scope of protection of the present disclosure.

Provided is a discovery method of cotton salt-tolerant genes based on combined transcriptome and proteome analysis, wherein the cotton salt-tolerant genes include cotton salt-tolerant gene Gh_D10G0907 and cotton salt-tolerant gene Gh_D11G0978 which are respectively located on cotton chromosomes D10 and D11.

Where, the cDNA sequence information of the cotton salt-tolerant gene Gh_D10G0907 and the cotton salt-tolerant gene Gh_D11G0978 is as follows:

>Gh_D10G0907.1
ATGGCTTCAGTGAATCTGGGCTCTTTGGTTCAATCATACTCCATTTTCA
ACCAAGCTTCC
AGAAACAATTCCAAGTCTTTTCCTTTACCCAGATCCTTTCCCTTCAATC
CCCTCAAGAAT
ATCTCATCTTCAACTCCCAAAAGACGTAGCTTTACATCTCTTCCTTCGA
TATCCTCAGTC
CTTACCAAAGAAGACGCGGTCATGGAAGAAGACCAAGACCCTCAATTAC
CAAGTTTTGAT
TTCAAATCATTCATGATACACAAGGGTAATGCAGTTAACCAAGCCCTGG
ACTCGGCTGTT
CCACTCCGTGACCCTGTTAAAATCCATGAAGCAATGCGTTACTCCCTTT
TAGCCGGTGGC
AAAAGGGTCCGCCCAGTTCTTTGTTTGGCTGCTTGTGACCTTGTTGGTG
GCAAAGAATCC
ATGGTTATGCCAGCAGCTTGTGCTCTTGAAATGATCCACACCATGTCTT
TAGTCCACGAT
GATCTTCCTTGCATGGACAACGATGATCTTCGTAGAGGGAAACCAACTA
ACCACAAAGTT
TATGGTGAAGATATAGCTGTGTTAGCAGGGGATGCTCTTTTAGCCTTTT
CGTTTGAACAT
ATAGCTGTATCCACAGTTGGTGTCACACCTGATAGGATTGTAAGAGCAA
TTGGGGAATTA
GCTAAATCTATTGGGGCTGAAGGGTTGGCGGCTGGTCAAGTTGTGGATA
TAACCAGTGAG
GGTCTAACCAATGTGGGGTTGGATCATTTAGAATTCATTCATGTTCATA
AAACTGCTCCA
TTGCTTGAAGCAGCTGCGGTTTTAGGGGCTATTCTTGGAGGTGGACATG
ATGAAGATGTG
GAAAAGTTGAGGAAATTTGCAAGGAATATTGGGCTTTTATTTCAAGTTG
TGGATGATATT
CTTGATGTAACAAAGTCATCTAAAGAATTAGGGAAGACTGCAGGGAAAG
ATTTGGTGGCT
GATAAAGTGACTTATCCTAAATCGATGGGGATAAACAAATCAAAGGAGT
TTGCAGAGAAG
TTGAAGAGTGATGCATTAGAGTTGCTTCAAGGGTTTGATCCTGAGAAAT
CTACCCCCTTA
ATTGCTTTAGCTAATTATATAGCTTACAGGCAAAATTAG
>Gh_D11G0978.1
ATGTCGCAGTTGTTGGAGAAGGCGAAGGACTTCGTGGTGGATAAGGTGG
CCAACATAAAG
AAGCCGGAGGCTAGTGTCTCGGACGTTGATCTGAAACATGTGAGCCGTG
AGTGCGTCGAG
TATGGCGCTAAGGTCTCTGTCTCCAACCCCTACAGCCATTCCATCCCCA
TTTGTGAGATC
TCTTACAATTTCAAAAGTGCTGGAAGAGGGATAGCATCAGGGACAATAC
CAGACCCGGGG
TCATTGAAAGCCGGCGACACAACGATGCTGGACGTGCCAGTGAAGGTGC
CGTATAACATC
TTAGTAAGCTTGGCAAAGGATATTGGTGCAGATTGGGACATTGACTATG
AATTGGAATTG
GGTCTCACCATTGATCTTCCTATCATGGGGAACTTCACTATCCCTCTCT
CTCAGAAAGGA
GAGATCAAGCTTCCTACTCTCAGTGACATCTTTTAG

Specifically, the present disclosure used a cotton salt-tolerant variety Tongyan No. 1 as a material for combined transcriptome and proteome analysis under salt stress and control to preliminarily determine salt-tolerant candidate genes, and RT-qPCR expression analysis was performed on salt-tolerant differential genes, and finally functional validation was conducted in cotton. Specific steps were as follows:

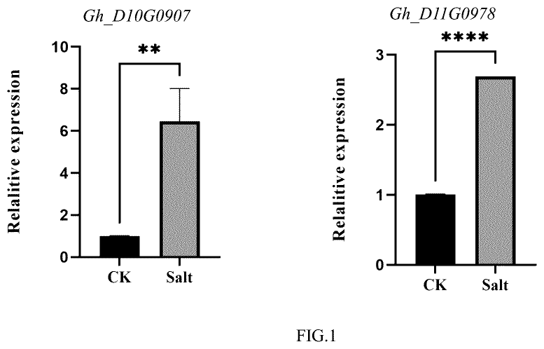

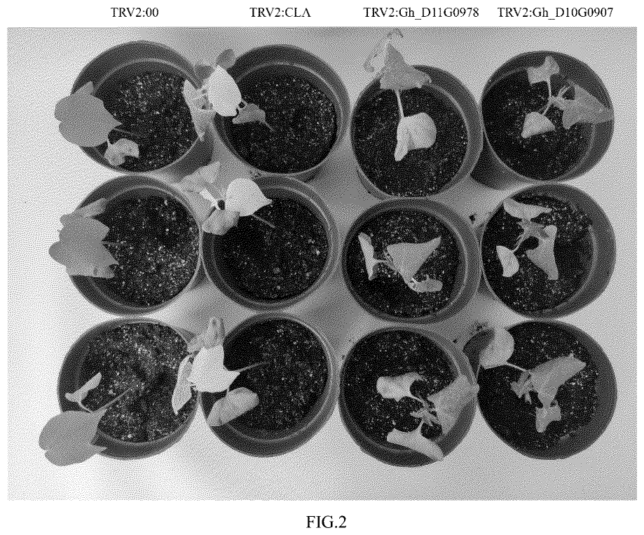

Referring to FIGS. 1-2, a discovery method of cotton salt-tolerant genes based on combined transcriptome and proteome analysis comprised the following steps:

Step 1, transcriptome and proteome sequencing and combined analysis

Salt-tolerant cotton varieties were planted, and 6 seedlings with good growth status and similar morphology were selected and divided into two groups when the cotton seedlings grow to the four-leaf stage under the same environment, wherein one group was a control group, and the other group was an experimental group. The experimental group was irrigated with 200 ml of 250 mmol/l NaCl solution at 5 pm every day; the control group was irrigated with an equal amount of distilled water. After two days, main leaves of three seedlings in the control group were respectively marked as CK1, CK2 and CK3, and main leaves of three seedlings in the experimental group were also respectively marked as Salt1, Salt2 and Slat3. Finally, 6 samples were frozen in dry ice, and then RNA was respectively extracted for transcriptome sequencing, and proteins were respectively extracted for proteome sequencing.

A total of 24 commonly differentially expressed genes were obtained by combined transcriptome and proteome analysis, and all the 24 genes exhibited significant differential expression in transcriptome and proteome results. Two significantly differentially-expressed genes Gh_D10G0907 and Gh_D11G0978 are obtained through annotation and alignment of target genes, comparison of expression difference before and after salt stress and summarization of gene functions, and their homologous genes in other species are found with significant salt tolerance. Glycerol-3-phosphate and ADP-glucose were synthesized to obtain glycerol glucose phosphate by using Gh_D10G0907 as glycerol glucoside phosphate synthase (GGPS) which was a known salt-tolerant gene in algae and microorganisms. The Gh_D10G0908 gene was a class of late embryogenesis abundant (LEA) protein related genes, also participated in regulating permeation and was a class of known stress genes, which was found to have slat-tolerant drought-tolerant and cold-tolerant functions in multiple crops such as corns and wheat. Hence, these two genes were determined as target genes for salt-tolerant function validation.

Step 2, expression mode validation of salt-tolerant genes

Step 2.1, primer design and synthesis were performed on the above salt-tolerant candidate genes by using Primer5, reverse transcription of RNA into cDNA was performed, and expression mode validation was performed by utilizing real-time quantitative PCR;

Step 2.2, gene cloning of candidate genes and functional validation based on virus-induced gene silencing (VIGS) were performed in cotton. Gh_D10G0907 had 372 full-length 1119 bp coding amino acids, and belonged to GGPS family; Gh_D10G0908 had 151 full-length 456 bp coding amino acids, and belonged to LEA family. Both of the two families had salt-tolerance related validation in different species. The gene-silenced plants of Gh_D10G0907 and Gh_D10G0908 were obtained by using a VIGS technology, and the silenced plants exhibited early wilting with a greater degree of salt damage phenotype after salt treatment. Moreover, the silenced plants showed higher levels of reactive oxygen species (ROS) than the control group. Therefore, the two genes were believed to have pivotal role in salt stress in upland cotton.

A total of 24 commonly differentially expressed genes were obtained by combined transcriptome and proteome analysis, and all the 24 genes exhibited significant differential expression in transcriptome and proteome results. These 24 genes were used as candidate genes for studying a response mechanism of upland cotton under salt stress. Two significantly differentially-expressed genes Gh_D10G0907 and Gh_D11G0978 are obtained through annotation and alignment of target genes, comparison of expression difference before and after salt stress and summarization of gene functions, and their homologous genes in other species are found with significant salt tolerance. Glycerol-3-phosphate and ADP-glucose were synthesized to obtain glycerol glucose phosphate by using Gh_D10G0907 as glycerol glucoside phosphate synthase (GGPS) which was a known salt-tolerant gene in algae and microorganisms. The Gh_D10G0908 gene was a class of late embryogenesis abundant (LEA) protein related genes, also participated in regulating permeation and was a class of known stress genes, which was found to have slat-tolerant drought-tolerant and cold-tolerant functions in multiple crops such as corns and wheat. Hence, these two genes were determined as target genes for salt-tolerant function validation. By RT-qPCR expression analysis (FIG. 1) and VIGS function validation (FIG. 2), it was indicated that overexpression of the two genes improved salt tolerance.

In summary, the cotton salt-tolerant genes Gh_D10G0907 and Gh_D11G0978 obtained according to the present disclosure are respectively located on cotton chromosomes D10 and D11. The genes can be transferred to a cotton material with excellent agronomic traits through molecule marker-assisted breeding, and then cotton materials or varieties with salt tolerance and excellent agronomic traits are developed.

The description and practice disclosed in the present disclosure are readily thought and understood by persons of ordinary skill in the art, and several modifications and improvements can also be made without departing from the principle of the present disclosure. Thus, modifications or improvements made without departing from the spirit of the present disclosure should also be deemed as the scope of protection of the present disclosure.

1. A discovery method of cotton salt-tolerant genes based on combined transcriptome and proteome analysis, wherein the cotton salt-tolerant genes comprise cotton salt-tolerant gene Gh_D10G0907 and cotton salt-tolerant gene Gh_D11G0978 which are respectively located on cotton chromosomes D10 and D11;
the cDNA sequence information of the cotton salt-tolerant gene Gh_D10G0907 and the cotton salt-tolerant gene Gh_D11G0978 is as follows:
>Gh_D10G0907.1
(SEQ ID NO: 1)
ATGGCTTCAGTGAATCTGGGCTCTTTGGTTCAATCATACTCCATTTTCA
ACCAAGCTTCC
(SEQ ID NO: 2)
AGAAACAATTCCAAGTCTTTTCCTTTACCCAGATCCTTTCCCTTCAATC
CCCTCAAGAAT
(SEQ ID NO: 3)
ATCTCATCTTCAACTCCCAAAAGACGTAGCTTTACATCTCTTCCTTCGA
TATCCTCAGTC
(SEQ ID NO: 4)
CTTACCAAAGAAGACGCGGTCATGGAAGAAGACCAAGACCCTCAATTAC
CAAGTTTTGAT
(SEQ ID NO: 5)
TTCAAATCATTCATGATACACAAGGGTAATGCAGTTAACCAAGCCCTGG
ACTCGGCTGTT
(SEQ ID NO: 6)
CCACTCCGTGACCCTGTTAAAATCCATGAAGCAATGCGTTACTCCCTTT
TAGCCGGTGGC
(SEQ ID NO: 7)
AAAAGGGTCCGCCCAGTTCTTTGTTTGGCTGCTTGTGACCTTGTTGGTG
GCAAAGAATCC
(SEQ ID NO: 8)
ATGGTTATGCCAGCAGCTTGTGCTCTTGAAATGATCCACACCATGTCTT
TAGTCCACGAT
(SEQ ID NO: 9)
GATCTTCCTTGCATGGACAACGATGATCTTCGTAGAGGGAAACCAACTA
ACCACAAAGTT
(SEQ ID NO: 10)
TATGGTGAAGATATAGCTGTGTTAGCAGGGGATGCTCTTTTAGCCTTTT
CGTTTGAACAT
(SEQ ID NO: 11)
ATAGCTGTATCCACAGTTGGTGTCACACCTGATAGGATTGTAAGAGCAA
TTGGGGAATTA
(SEQ ID NO: 12)
GCTAAATCTATTGGGGCTGAAGGGTTGGCGGCTGGTCAAGTTGTGGATA
TAACCAGTGAG
(SEQ ID NO: 13)
GGTCTAACCAATGTGGGGTTGGATCATTTAGAATTCATTCATGTTCATA
AAACTGCTCCA
(SEQ ID NO: 14)
TTGCTTGAAGCAGCTGCGGTTTTAGGGGCTATTCTTGGAGGTGGACATG
ATGAAGATGTG
(SEQ ID NO: 15)
GAAAAGTTGAGGAAATTTGCAAGGAATATTGGGCTTTTATTTCAAGTTG
TGGATGATATT
(SEQ ID NO: 16)
CTTGATGTAACAAAGTCATCTAAAGAATTAGGGAAGACTGCAGGGAAAG
ATTTGGTGGCT
(SEQ ID NO: 17)
GATAAAGTGACTTATCCTAAATCGATGGGGATAAACAAATCAAAGGAGT
TTGCAGAGAAG
(SEQ ID NO: 18)
TTGAAGAGTGATGCATTAGAGTTGCTTCAAGGGTTTGATCCTGAGAAAT
CTACCCCCTTA
(SEQ ID NO: 19)
ATTGCTTTAGCTAATTATATAGCTTACAGGCAAAATTAG
>Gh_D11G0978.1
(SEQ ID NO: 20)
ATGTCGCAGTTGTTGGAGAAGGCGAAGGACTTCGTGGTGGATAAGGTGG
CCAACATAAAG
(SEQ ID NO: 21)
AAGCCGGAGGCTAGTGTCTCGGACGTTGATCTGAAACATGTGAGCCGTG
AGTGCGTCGAG
(SEQ ID NO: 22)
TATGGCGCTAAGGTCTCTGTCTCCAACCCCTACAGCCATTCCATCCCCA
TTTGTGAGATC
(SEQ ID NO: 23)
TCTTACAATTTCAAAAGTGCTGGAAGAGGGATAGCATCAGGGACAATAC
CAGACCCGGGG
(SEQ ID NO: 24)
TCATTGAAAGCCGGCGACACAACGATGCTGGACGTGCCAGTGAAGGTGC
CGTATAACATC
(SEQ ID NO: 25)
TTAGTAAGCTTGGCAAAGGATATTGGTGCAGATTGGGACATTGACTATG
AATTGGAATTG
(SEQ ID NO: 26)
GGTCTCACCATTGATCTTCCTATCATGGGGAACTTCACTATCCCTCTCT
CTCAGAAAGGA
(SEQ ID NO: 27)
GAGATCAAGCTTCCTACTCTCAGTGACATCTTTTAG;
wherein the method comprises the following steps:
Step 1, transcriptome and proteome sequencing and combined analysis;
salt-tolerant cotton varieties are planted, and 6 seedlings with good growth status and similar morphology are selected and divided into two groups when the cotton seedlings grow to the four-leaf stage under the same environment, wherein one group is a control group, and the other group is an experimental group, the experimental group is irrigated with 200 ml of 250 mmol/l NaCl solution at 5 pm every day; the control group is irrigated with an equal amount of distilled water, after two days, main leaves of three seedlings in the control group are respectively marked as CK1, CK2 and CK3, and main leaves of three seedlings in the experimental group are also respectively marked as Salt1, Salt2 and Slat3, finally, 6 samples are frozen in dry ice, and then RNA is respectively extracted for transcriptome sequencing, and proteins are respectively extracted for proteome sequencing;
a total of 24 commonly differentially expressed genes are obtained by combined transcriptome and proteome analysis, and all the 24 genes exhibit significant differential expression in transcriptome and proteome results; two significantly differentially-expressed genes Gh_D10G0907 and Gh_D11G0978 are obtained through annotation and alignment of target genes, comparison of expression difference before and after salt stress and summarization of gene functions, and the two genes are determined as target genes for salt-tolerant function validation;
Step 2, expression mode validation of salt-tolerant genes;
Step 2.1, primer design and synthesis are performed on the above salt-tolerant candidate genes by using Primer5, reverse transcription of RNA into cDNA is performed, and expression mode validation is performed by utilizing real-time quantitative PCR;
Step 2.2, gene cloning of candidate genes and functional validation based on virus-induced gene silencing (VIGS) are performed in cotton, Gh_D10G0907 has 372 full-length 1119 bp coding amino acids, and belongs to GGPS family; Gh_D10G0908 has 151 full-length 456 bp coding amino acids, and belongs to LEA family; both of the two families have salt-tolerance related validation in different species; the gene-silenced plants of Gh_D10G0907 and Gh_D10G0908 are obtained by using a VIGS technology, the silenced plants exhibit early wilting with a greater degree of salt damage phenotype after salt treatment; moreover, the silenced plants show higher levels of reactive oxygen species (ROS) than the control group; and therefore the two genes are believed to have pivotal role in salt stress in upland cotton.

2. (canceled)

3. (canceled)

4. Use of the cotton salt-tolerant gene Gh_D10G0907 and the cotton salt-tolerant gene Gh_D11G0978 obtained by using the discovery method according to claim 1 in molecular marker-assisted breeding of cotton varieties.


SEQUENCE LISTING (27 sequences)

SEQ ID NO: 1 - DNA, 60 bp, synthetic construct
  source 1..60: mol_type = other DNA; note = cotton salt-tolerant gene
atggcttcagtgaatctgggctctttggttcaatcatactccattttcaaccaagcttcc

SEQ ID NO: 2 - DNA, 60 bp, synthetic construct
  source 1..60: mol_type = other DNA; note = cotton salt-tolerant gene
agaaacaattccaagtcttttcctttacccagatcctttcccttcaatcccctcaagaat

SEQ ID NO: 3 - DNA, 60 bp, synthetic construct
  source 1..60: mol_type = other DNA; note = cotton salt-tolerant gene
atctcatcttcaactcccaaaagacgtagctttacatctcttccttcgatatcctcagtc

SEQ ID NO: 4 - DNA, 60 bp, synthetic construct
  source 1..60: mol_type = other DNA; note = cotton salt-tolerant gene
cttaccaaagaagacgcggtcatggaagaagaccaagaccctcaattaccaagttttgat

SEQ ID NO: 5 - DNA, 60 bp, synthetic construct
  source 1..60: mol_type = other DNA; note = cotton salt-tolerant gene
ttcaaatcattcatgatacacaagggtaatgcagttaaccaagccctggactcggctgtt

SEQ ID NO: 6 - DNA, 60 bp, synthetic construct
  source 1..60: mol_type = other DNA; note = cotton salt-tolerant gene
ccactccgtgaccctgttaaaatccatgaagcaatgcgttactcccttttagccggtggc

SEQ ID NO: 7 - DNA, 60 bp, synthetic construct
  source 1..60: mol_type = other DNA; note = cotton salt-tolerant gene
aaaagggtccgcccagttctttgtttggctgcttgtgaccttgttggtggcaaagaatcc

SEQ ID NO: 8 - DNA, 60 bp, synthetic construct
  source 1..60: mol_type = other DNA; note = cotton salt-tolerant gene
atggttatgccagcagcttgtgctcttgaaatgatccacaccatgtctttagtccacgat

SEQ ID NO: 9 - DNA, 60 bp, synthetic construct
  source 1..60: mol_type = other DNA; note = cotton salt-tolerant gene
gatcttccttgcatggacaacgatgatcttcgtagagggaaaccaactaaccacaaagtt

SEQ ID NO: 10 - DNA, 60 bp, synthetic construct
  source 1..60: mol_type = other DNA; note = cotton salt-tolerant gene
tatggtgaagatatagctgtgttagcaggggatgctcttttagccttttcgtttgaacat

SEQ ID NO: 11 - DNA, 60 bp, synthetic construct
  source 1..60: mol_type = other DNA; note = cotton salt-tolerant gene
atagctgtatccacagttggtgtcacacctgataggattgtaagagcaattggggaatta

SEQ ID NO: 12 - DNA, 60 bp, synthetic construct
  source 1..60: mol_type = other DNA; note = cotton salt-tolerant gene
gctaaatctattggggctgaagggttggcggctggtcaagttgtggatataaccagtgag

SEQ ID NO: 13 - DNA, 60 bp, synthetic construct
  source 1..60: mol_type = other DNA; note = cotton salt-tolerant gene
ggtctaaccaatgtggggttggatcatttagaattcattcatgttcataaaactgctcca

SEQ ID NO: 14 - DNA, 60 bp, synthetic construct
  source 1..60: mol_type = other DNA; note = cotton salt-tolerant gene
ttgcttgaagcagctgcggttttaggggctattcttggaggtggacatgatgaagatgtg

SEQ ID NO: 15 - DNA, 60 bp, synthetic construct
  source 1..60: mol_type = other DNA; note = cotton salt-tolerant gene
gaaaagttgaggaaatttgcaaggaatattgggcttttatttcaagttgtggatgatatt

SEQ ID NO: 16 - DNA, 60 bp, synthetic construct
  source 1..60: mol_type = other DNA; note = cotton salt-tolerant gene
cttgatgtaacaaagtcatctaaagaattagggaagactgcagggaaagatttggtggct

SEQ ID NO: 17 - DNA, 60 bp, synthetic construct
  source 1..60: mol_type = other DNA; note = cotton salt-tolerant gene
gataaagtgacttatcctaaatcgatggggataaacaaatcaaaggagtttgcagagaag

SEQ ID NO: 18 - DNA, 60 bp, synthetic construct
  source 1..60: mol_type = other DNA; note = cotton salt-tolerant gene
ttgaagagtgatgcattagagttgcttcaagggtttgatcctgagaaatctaccccctta

SEQ ID NO: 19 - DNA, 39 bp, synthetic construct
  source 1..39: mol_type = other DNA; note = cotton salt-tolerant gene
attgctttagctaattatatagcttacaggcaaaattag

SEQ ID NO: 20 - DNA, 60 bp, synthetic construct
  source 1..60: mol_type = other DNA; note = cotton salt-tolerant gene
atgtcgcagttgttggagaaggcgaaggacttcgtggtggataaggtggccaacataaag

SEQ ID NO: 21 - DNA, 60 bp, synthetic construct
  source 1..60: mol_type = other DNA; note = cotton salt-tolerant gene
aagccggaggctagtgtctcggacgttgatctgaaacatgtgagccgtgagtgcgtcgag

SEQ ID NO: 22 - DNA, 60 bp, synthetic construct
  source 1..60: mol_type = other DNA; note = cotton salt-tolerant gene
tatggcgctaaggtctctgtctccaacccctacagccattccatccccatttgtgagatc

SEQ ID NO: 23 - DNA, 60 bp, synthetic construct
  source 1..60: mol_type = other DNA; note = cotton salt-tolerant gene
tcttacaatttcaaaagtgctggaagagggatagcatcagggacaataccagacccgggg

SEQ ID NO: 24 - DNA, 60 bp, synthetic construct
  source 1..60: mol_type = other DNA; note = cotton salt-tolerant gene
tcattgaaagccggcgacacaacgatgctggacgtgccagtgaaggtgccgtataacatc

SEQ ID NO: 25 - DNA, 60 bp, synthetic construct
  source 1..60: mol_type = other DNA; note = cotton salt-tolerant gene
ttagtaagcttggcaaaggatattggtgcagattgggacattgactatgaattggaattg

SEQ ID NO: 26 - DNA, 60 bp, synthetic construct
  source 1..60: mol_type = other DNA; note = cotton salt-tolerant gene
ggtctcaccattgatcttcctatcatggggaacttcactatccctctctctcagaaagga

SEQ ID NO: 27 - DNA, 36 bp, synthetic construct
  source 1..36: mol_type = other DNA; note = cotton salt-tolerant gene
gagatcaagcttcctactctcagtgacatcttttag